(19) United States
(12) Patent Application Publication
(10) Pub. No.: US 2024/0180741 A1
(43) Pub. Date: Jun. 6, 2024

(54) OSTOMY BAG AND METHOD OF USING SAME

(71) Applicants: Brian Zimmerman, Orem, UT (US); Wes Hamman, Highland, UT (US)

(72) Inventors: Brian Zimmerman, Orem, UT (US); Wes Hamman, Highland, UT (US)

(21) Appl. No.: 18/061,505

(22) Filed: Dec. 5, 2022

(51) Int. Cl. A61F 5/445; A61F 5/44

(52) U.S. Cl. CPC A61F 5/445; A61F 5/4407

(57) ABSTRACT

An ostomy device includes a ostomy bag and a stoma port connector connected thereto about an opening in the ostomy bag and having an external connection surface configured for connection to an external surface of a stoma port which is operably disposed on a stoma of a patient requiring treatment. A removable closure mechanism is operably connected to the stoma port connector and operates between a first open position to provide communication through the stoma port connector and a second closed position to seal the ostomy bag preferably prior to removal of the ostomy device.

### FIELD OF INVENTION

The present invention relates to the field of draining fluid through artificial openings in a patient. More particularly, the invention relates to an improved ostomy bag for use in a stoma and method of using same.

### BACKGROUND OF THE INVENTION

A colostomy is a surgical operation in which the patient's colon is provided with an artificial opening for the drainage or evacuation of its contents. The external or visible portion of this opening, referred to as the stoma, is normally located on one side of the abdomen of the patient.

The contents of the colon are discharged from time to time through the stoma, and an ostomy device must be provided to collect the material thus discharged in as sanitary and unoffensive a manner as possible. To this end, the patient employs a disposable colostomy bag. The bag is typically formed of a flexible sheet material that is impervious to fluids, such as sheet rubber, rubberized fabric, thin plastic sheets or films, and the like, a conventional bag comprising two side wall panels joined together along the top, both sides, and bottom edges of the side walls. One of the side wall panels is formed with an opening adapted to receive a stoma port, the bag can be secured to the patient in a variety of ways, such as via adhesion or a connection device with one part connected to the skin of the patient immediately surrounding the stoma and a complementary part connected to the ostomy bag. When properly applied to the person, the colostomy bag provides a gas tight and fluid-tight receptacle for material discharged through the stoma.

It is obvious that in the passage of time the colostomy bag will become filled to its working capacity with material discharged through the stoma and must then be removed and replaced with a new colostomy bag. The frequency with which this must be done differs with the individual involved. However, in all cases in order to replace the bag the used ostomy bag must be removed from the patient in the manner dictated by the connection type. Another ostomy bag is then secured in place of the used ostomy bag and the used ostomy bag is discarded.

Due to the need for fairly frequent replacement of conventional colostomy bags the use of such bags, the process can be rather foul with the odor of gas which typically is allowed to escape. Currently, removal of the bag leads to leakage of fecal material and fluid from the ostomy bag before it can be disposed. This can also be an embarrassment to the patient.

In view of the problems and disadvantages inherent in conventional colostomy bags, there exists an urgent need for an ostomy device which will improve the removal and containment of gas, fluid and fecal material in the ostomy bag.

### SUMMARY OF THE INVENTION

It is an object to improve the method of draining artificial openings in a patient.

It is another object of the invention to provide an improved ostomy bag.

It is yet another object to provide an improved apparatus for interconnecting a ostomy bag and a stoma port.

Yet another object is to provide a solution in an ostomy bag which enables closure prior to removal of the bag prior to removal from the stoma port and avoid any leakage.

Accordingly, one embodiment of the invention is directed to a method of removing fecal material from a person requiring use of an ostomy bag, which includes the steps of:
(a) employing an ostomy device having an ostomy bag for containing fecal material and a stoma port connector, a patient having a stoma port configured for connection to an external surface of the stoma port connector and which is operably disposed in a stoma of a patient, and a removable closure mechanism which operates between a first open position to provide communication through the stoma port connector to the ostomy bag and a second closed position to seal the ostomy bag;
(b) operably attaching the stoma port connector to the stoma port in a manner to receive fecal therethrough;
(c) permitting the ostomy bag to fill with fluid and fecal material;
(d) placing the removable closure mechanism of the ostomy bag in closed position to block communication.

The step (d) can be prior to removing the ostomy bag, and include the step of
(e) removing said ostomy bag by disconnecting the stoma port connector from the stoma port.

Another embodiment is directed to an ostomy device. The ostomy bag includes a ostomy bag and a stoma port connector connected thereto about an opening in the ostomy bag and having an external connection surface configured for connection to an external surface of a stoma port which is operably disposed on a stoma of a patient requiring treatment. A removable closure mechanism operably connects to the stoma port connector and operates between a first open position to provide communication through the stoma port connector and a second closed position to seal the ostomy bag.

In a preferred embodiment, the closure mechanism includes a sliding door which sealably slides in and out of the open position and the closed position and is received by opposing tracks about the stoma port connector. Another embodiment includes a hinged door which sealably connects about the opening in and out of the open position and the closed position and is received about the opening of the stoma port connector.

Additional objects and advantages will be apparent to those skilled in the art when reading the following specification and drawings attached hereto.

### BRIEF DESCRIPTION OF THE DRAWINGS

FIG. 1 is a front perspective view of an embodiment of the invention in an open mode.

FIG. 2 is a side view of FIG. 1.

FIG. 3 is a front perspective view of the embodiment of FIG. 1 in a closed mode.

FIG. 4 is a side view of FIG. 3.

FIG. 5 is a front perspective view of another embodiment of the invention.

FIG. 6 is a side view of FIG. 5.

FIG. 7 is a front view of the embodiment of FIG. 6 with components separated in an open mode.

FIG. 8 is a front view of the embodiment of FIG. 6 with components in a partially open mode.

FIG. 9 is a side view of FIG. 8.

FIG. 10 is a front view of the embodiment of FIG. 6 with components separated in a closed mode.

FIG. 11 depicts a patient with a stoma.

FIG. 12 illustrates a side view of a stoma port in a patient.

### DETAILED DESCRIPTION OF THE PREFERRED EMBODIMENT

Referring now to the drawings, an ostomy device of the instant invention is generally referred to by the numeral 10. Like numerals refers to like parts.

The ostomy bag 10 includes a ostomy bag 12 and a stoma port connector 14, which can include a protruding ring, formed about an opening 16 in the bag 12 and has an external connection surface portion 18. The ostomy bag 12 is typically formed from a waterproof material which acts as a barrier to flatus gases. Examples of such materials include polyvinyl chloride (PVC), polyvinyl dichloride (PVDC)₅ ethylene vinyl alcohol and related materials and combinations thereof. A stoma port 20 is operably disposed on a stoma 22 of a patient P requiring treatment. The external connection surface portion 18 is configured for connection to an external surface 24 of the stoma port 20.

The stoma port connector 14 has a removable closure mechanism 28 which operates between a first open position to provide communication through the stoma port connector 14 and a second closed position which seals and prevents communication through the stoma port connector 14.

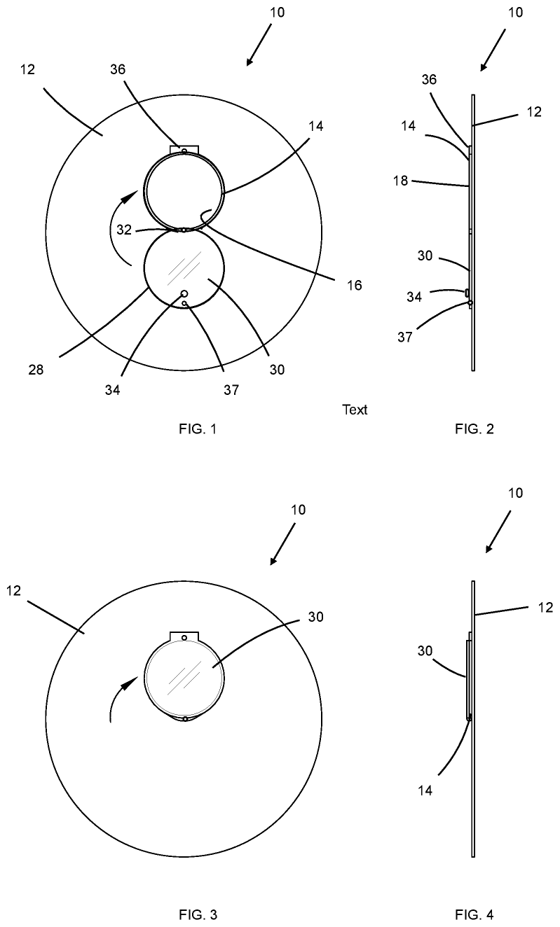

As can be seen in FIGS. 1-4, closure mechanism 28 includes a hinged door 30 which sealably connects about the stoma port connector 14 in and out of the open position and the closed position and is received about the stoma port connector 14. Adjacent the ring 15, there can be provided a movable junction 32 to permit the door 30 and ring 15 to sealably connect. It is envisioned the door 30 can be connected to a hinge via a plastic connection (made from polypropylene, polyethylene and polyamides such as “Nylon” for example). The door 30 can be provided with a protruding nub 34 which can be used to rotate the door 30 into position such that a nub serving as a latch 37 can be complementary connected to an opposing receiving surface 36 adjacent an opposing side of the ring 14. A latch 37 on the door 30 can further secure that the door 30 is sealably connected to the ring 14 preventing any leaking from occurring from the ostomy bag 12.

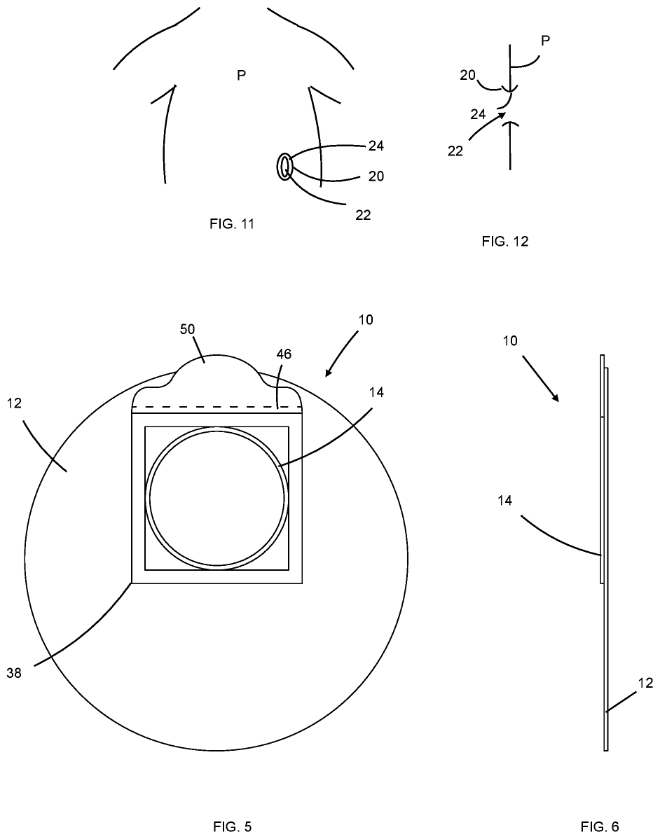

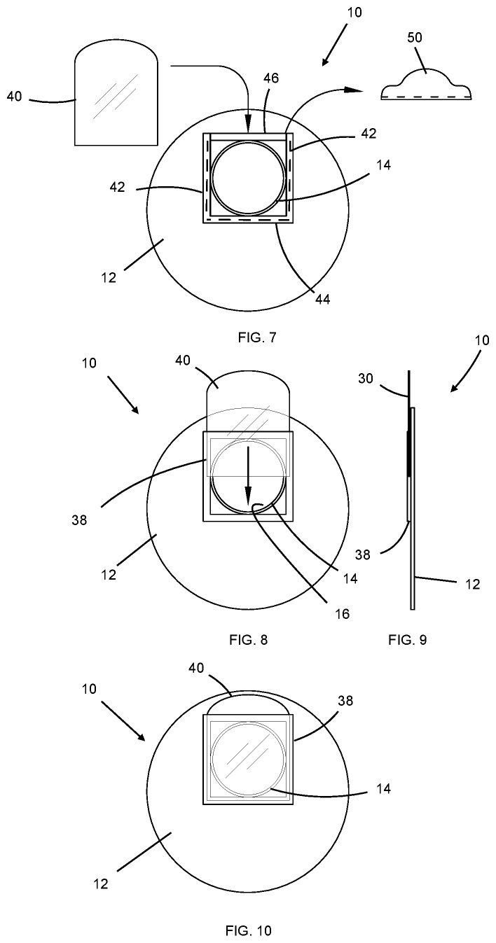

Another embodiment is depicted in FIGS. 5-10 and includes a removable closure mechanism 38 having a sliding door 40 which sealably slides in and out of the open position and the closed position of the stoma port connector (ring) 14. Here, disposed about the ring 14 are opposing tracks 42 having grooves running lengthwise which interconnect with a lower end portion 44 which can likewise have a groove co-aligning with the grooves of the tracks 42, wherein the grooves are depicted in dashed lines. An upper end portion 46 is also provided interconnecting the tracks 42. Upper end portion 46 has a slightly smaller profile to allow for friction seal and sliding receipt of a door 40 in the grooves. The door 40 is slidably sealably received by opposing tracks 42 and lower end portion 44 and frictionally is held in place against outer surface of the ring 14 to form a seal and prevent leakage of the fluid. The door 40 can be made of a plastic material which is relatively rigid.

A removable tab closure 50 can be provided on the ostomy bag 12 and extends out of the removable closure mechanism 38. With regards to the detachable tab 50, this tab 50 extends along the upper end 46. This tab 50 collectively with the tracks 42 and lower end 44 are sealably disposed on the patient P and seal the ostomy bag 12 to the patient P and not allow any fluid or any other material in/out of the stoma bag 12 about the connection. The tab 50 includes perforation 52 for removal and once the tab 50 is removed, it opens up the ostomy bag 12 at the upper end 46 to permit the door 40 (closure piece) to be slid down in the channel to close off the ostomy bag 12 opening and allow it to be removed. The tab closure 50 can be provided as a safety measure and removed prior to installation of the door 40. It is noted that closure mechanism 38 could optionally use adhesive tapes or other materials to maintain a seal of the same to the patient P, but the preferred embodiment provides an elegant means and it is envisioned can be provided with a self adhesive facing which can have a release liner to be removed prior to application.

In implementing the invention, there is provided a method of removing fecal material from a person requiring use of an ostomy bag. The method includes the steps of
(a) employing a stoma device having an ostomy bag for containing fluid material and a stoma port connector, a patient having a stoma port configured for connection to an external surface of the stoma port connector and which is operably disposed on the patient, a removable closure mechanism operably connects to the stoma port connector and operates between a first open position to provide communication through the stoma port connector to the ostomy bag and a second closed position which seals the port and closes the ostomy bag;
(b) operably attaching the stoma port connector to the stoma port in a manner to receive fluid and fecal material therethrough;
(c) permitting the ostomy bag to fill with fluid and fecal material; and
(d) placing the removable closure mechanism of the ostomy bag in closed position to block communication and seal the ostomy bag.

The method can further include that prior to the step (d) be characterized to be performed prior to removing the ostomy bag, and further includes the step
(e) removing the ostomy bag by disconnecting the stoma port connector from the stoma port.

The components of the ostomy bag 10 can be made of suitable plastic materials which are physiologically acceptable for the purposes intended, i.e., be made in analogy with and from materials conventionally used for the preparation of ostomy appliances. It may be desirable to employ biodegradable materials as is known in the art.

While there are shown and described herein specific forms of the invention, it will be readily apparent to those skilled in the art that the invention is not so limited, but is susceptible to various modifications and rearrangements in design and materials without departing from the spirit and scope of the invention. In particular, it should be noted that the present invention is subject to modification with regard to any dimensional relationships set forth herein and modifications in assembly, materials, size, shape, and use. For instance, there are numerous components described herein that can be replaced with equivalent functioning components to accomplish the objectives of the present invention.

What is claimed is:

1. An ostomy device, which includes:
an ostomy bag and a stoma port connector connected thereto about an opening in said ostomy bag and having an external connection surface configured for connection to an external surface of a stoma port which is operably disposed on a stoma of a patient requiring treatment; and
a movable closure mechanism operably connected to said stoma port connector which operates between a first open position to provide communication through said stoma port connector and a second closed position to seal said ostomy bag.

2. The ostomy bag of claim 1, wherein said closure mechanism includes a sliding door which sealably slides in and out of said open position and said closed position and is received by opposing tracks about said stoma port connector.

3. The ostomy bag of claim 1, wherein said closure mechanism includes a hinged door which moves in and out of said open position and said closed position to sealably connect against said stoma port connector.

4. The ostomy bag of claim 3, wherein said door includes a protruding nub for assisting on moving said hinged door.

5. The ostomy bag of claim 2, wherein said closure mechanism includes a removable seal portion operably connected to said closure mechanism adjacent said tracks and to seal communication thereto and is removable to permit insertion of said sliding door.

6. A method of removing fecal material from a person requiring use of a ostomy bag, which includes the steps of:
(a) employing an ostomy device having a ostomy bag for containing fluid and fecal material and a stoma port connector, a patient having a stoma port configured for connection to an external surface of the stoma port connector and which is operably disposed on the patient, and a removable closure mechanism which operates between a first open position to provide communication through the stoma port connector to the ostomy bag and a second closed position which seals the port and prevents communication between to the ostomy bag;
(b) operably attaching the stoma port connector to the stoma port in a manner to receive fecal therethrough;
(c) permitting the ostomy bag to fill with fluid and fecal material; and
(d) placing the removable closure mechanism of the ostomy bag in closed position to block communication.

7. The method of claim 6, which is further characterized that the step (d) can be prior to removing the ostomy bag; and include the step of (e) removing the ostomy bag by disconnecting the stoma port connector from the stoma port.
